(19) United States
(12) Patent Application Publication
(10) Pub. No.: US 2021/0333280 A1
(43) Pub. Date: Oct. 28, 2021

(54) METHODS RELATED TO THE DIAGNOSIS OF PROSTATE CANCER

(71) Applicant: YatHing Biotechnology Company Limited, Hong Kong (CN)

(72) Inventor: Yin Lam Kenneth NG, Hong Kong (CN)

(21) Appl. No.: 17/302,078

(22) Filed: Apr. 23, 2021

Related U.S. Application Data
(60) Provisional application No. 63/014,178, filed on Apr. 23, 2020.

(51) Int. Cl. G01N 33/574; G16H 50/20; G16H 50/30

(52) U.S. Cl. CPC G01N 33/57434; G01N 33/57488; A61B 10/0241; G16H 50/30; G16H 50/20

(57) ABSTRACT

Disclosed herein are methods of determining the risk of developing prostate cancer in a subject or determining whether a subject suffers from prostate cancer, the method comprising measuring the level of one or more polyamines in a fluid sample obtained from the subject, measuring at a variable selected from the group consisting of age, prostate volume (PV), prostate-specific antigen (PSA), digital rectal examination (DRE), and combinations thereof. Also disclosed herein are methods of determining the risk of developing prostate cancer in a subject or determining whether a subject suffers from prostate cancer, the method comprising obtaining a score value based on previously disclosed variables to predict the likelihood of the subject developing or having prostate cancer.

### CROSS-REFERENCE TO RELATED APPLICATIONS

This application claims the benefit of priority of U.S. provisional patent application No. 63/014,178, filed 23 Apr. 2020, the contents of it being hereby incorporated by reference in its entirety for all purposes.

### FIELD OF THE INVENTION

The present disclosure relates to the field of molecular biology and bioinformatics. More specifically, the present disclosure relates to a method determining the risk of developing prostate cancer in a subject or determining whether a subject suffers from prostate cancer.

### BACKGROUND OF THE INVENTION

In the United States, by 2021, there will be an estimated over 1.8 million new cancer cases and over 600 thousand cancer deaths. According to American Cancer Society, it is estimated that there will be over 33000 deaths from prostate cancer, which account for 10% of all male cancer deaths in the United States. This high incidence of, and death rate due to, prostate cancer indicates the presence of an important public health concern.

Since its approval by the American Food and Drug Administration (FDA) in 1986, screening and early detection of prostate cancer has primarily been done by detecting prostate-specific antigen (PSA) in serum. Elevated prostate-specific antigen levels are typically regarded as an abnormal result and are treated as an indication of requiring further follow-up tests to confirm diagnosis of prostate cancer, such as for example, digital rectal examination (DRE), magnetic resonance imaging (MRI) and, in certain cases, a prostate biopsy. However, the prostate-specific antigen to prostate cancer has limited diagnostic performance within the range 4.0-10.0 ng/mL and some reported the range 4.0-20.0 ng/mL for Asians. At this range, the use of prostate-specific antigen has been shown to result in high false-positive rates, over-diagnosis of low-risk tumours, and in subsequent, unnecessary, and invasive biopsies. Therefore, there is an unmet need for the development of a method of detecting prostate cancer that does not rely solely on the detection of prostate-specific antigen.

### SUMMARY

In one aspect, the present disclosure refers to a method of determining the risk of developing prostate cancer in a subject or determining whether a subject suffers from prostate cancer, the method comprising measuring the level of one or more polyamines in a fluid sample obtained from the subject, measuring at a variable selected from the group consisting of age, prostate volume (PV), prostate-specific antigen (PSA), digital rectal examination (DRE), and combinations thereof; comparing the level of the one of more polyamines and the at least one variable to a control; wherein a decrease or increase in the level of the one or more polyamines compared to the control indicates that the subject is at risk of developing, or suffers from, prostate cancer; wherein a decrease in prostate volume and/or an increase in prostate-specific antigen (PSA) compared to the control indicates that the subject is at risk of developing, or suffers from, prostate cancer; wherein a positive digital rectal examination result indicates that the subject is at risk of developing, or suffers from, prostate cancer; wherein the one or more polyamines is selected from the group consisting of spermine, spermidine and putrescine.

In another aspect, the present disclosure refers to a method of determining the risk of developing prostate cancer in a subject or determining whether a subject suffers from prostate cancer, the method comprising measuring the level of one or more polyamines in a fluid sample obtained from the subject, measuring a variable selected from the group consisting of age, prostate volume (PV), prostate-specific antigen (PSA), digital rectal examination (DRE), and combinations thereof; obtaining a score value based on the level of the one or more polyamines measured herein and the at least one variable measured herein to predict the likelihood of the subject developing or having prostate cancer; wherein an increase in score value indicates that the subject is at an increased risk of developing, or suffers from, prostate cancer.

In yet another aspect, the present disclosure refers to a kit for use according to the method disclosed herein.

### BRIEF DESCRIPTION OF THE DRAWINGS

The invention will be better understood with reference to the detailed description when considered in conjunction with the non-limiting examples and the accompanying drawings, in which:

FIG. 1 shows Table 1 that shows the descriptive statistics of 162 subjects.

FIG. 2 shows Table 2 that shows the odd ratios and p-value from logistic regression using urinary polyamines as predictors of the 162-subject dataset.

FIG. 3 shows Table 3 that shows the coefficients and its 95% confidence interval of the logistic regression using log 2 transformed normalised spermine and log 2 transformed normalised putrescine as predictors of prostate cancer of the 162-subject dataset.

FIG. 4 shows Table 4 that shows the coefficients and its 95% confidence interval of the logistic regression for model 1 of the 162-subject dataset.

FIG. 5 shows Table 5 that shows the coefficients and its 95% confidence interval of the logistic regression for model 2 of the 162-subject dataset.

FIG. 6 shows the receiver operating characteristics (ROC) of normalized spermine on positive biopsy results detection of the 162-subject dataset.

FIG. 7 shows the receiver operating characteristics (ROC) of normalized spermidine on positive biopsy results detection of the 162-subject dataset.

FIG. 8 shows the receiver operating characteristics (ROC) of normalized putrescine on positive biopsy results detection of the 162-subject dataset.

FIG. 9 shows the receiver operating characteristics (ROC) of model 1 on positive biopsy results detection of the 162-subject dataset.

FIG. 10 shows the receiver operating characteristics (ROC) of model 2 on positive biopsy results detection of the 162-subject dataset.

FIG. 11 shows the results of the internal validation of the Spermine Risk Score for any grade prostate cancer and high-grade prostate cancer of the 600-subject dataset.

FIG. 12 shows decision curve analyses (DCA) for any grade prostate cancer (PCa) and high-grade prostate cancer (HGPCa) of the 600-subject dataset.

FIG. 13 shows Table 6 that shows the baseline characteristics of the cancer and non-cancer patients of the 600-subject dataset.

FIG. 14 shows Table 7 that shows normalized Spermine and risk of Prostate cancer (PCa) and high-grade PCa (HGPCa) of the 600-subject dataset.

FIG. 15 shows Table 8 that shows the univariate and multivariate analyses for prediction of PCa and HGPCa (ISUP grade 2 or above cancer) of the 600-subject dataset.

FIG. 16 shows Table 9 that shows area under the curve (AUC) of the calculated probabilities of the different predictive models of the 600-subject dataset.

### DEFINITIONS

As used herein, the terms “prostate cancer” and “high-grade prostate cancer” refer to cancers of the prostate. The prostate is a gland in the male reproductive system that surrounds the urethra just below the bladder. Most prostate cancers are slow growing. The cancer may metastasize to other areas of the body, for example but not limited to, the bones and lymph nodes.

There are 2 types of staging for prostate cancer: clinical staging and pathological staging.

Clinical staging is based on the results of digital rectal examination, prostate-specific antigen testing, and Gleason score. These factors help determine whether x-rays, bone scans, CT scans, or MRIs are subsequently needed.

Pathologic staging is based on information obtained from biopsies, which can be obtained via surgery. The surgery often includes the removal of the entire prostate and some lymph nodes. Examination of the removed lymph nodes can provide more information for pathologic staging.

Prostate cancer is also given a grade called a Gleason score. This score is based on how similar or different the cancer compared to healthy tissue in histological or histopathological analysis. Less aggressive tumours generally look more like healthy tissue. Metastatic tumours are aggressive and less like healthy tissue.

The Gleason scoring system is the most common prostate cancer grading system used. A pathologist looks at how the cancer cells are arranged in the prostate and assigns a score on a scale of 1 to 5. Cancer cells that look similar to healthy cells receive a low score. Cancer cells that look less like healthy cells or look more aggressive receive a higher score. To assign the grades, the pathologist determines the main pattern of cell growth, which is the most frequent pattern seen from the samples, and then looks for next-most frequent pattern observed from the samples. These are the primary and secondary grade. The grades are added together to come up with an overall score between 6 and 10.

Gleason scores of 6 or lower are low-grade cancer, the cancer cells look moderately like normal cells. Cancer cells of Gleason score of 7 look moderately to poorly like normal prostate cells, and a score of 8, 9, or 10 is a high-grade cancer, the cancer cells are poorly differentiated as a normal cell. A lower grade cancer grows more slowly and is less likely to spread than a high-grade cancer. Thus, as used herein, the term “high-grade prostate cancer” refers to a prostate cancer with a Gleason score of at least 7.

Another system used to grade the severity of prostate cancers is known as the International Society of Urological Pathology (ISUP) Grade Groups. Compared to the Gleason score, the ISUP grade group has less grades (1 to 5) but is likewise based on pathological analysis of biopsy samples. The table provided below provides a correlation between the ISUP grade group and the Gleason score.

| Risk group | ISUP Grade Group | Gleason Score |
|---|---|---|
| Low | Grade Group 1 | Gleason ≤6 |
| Intermediate favourable | Grade Group 2 | Gleason 7 (3 + 4) |
| Intermediate unfavourable | Grade Group 3 | Gleason 7 (4 + 3) |
| High | Grade Group 4 | Gleason 8 |
| High | Grade Group 5 | Gleason 9 to 10 |

As used herein, the term “negative predictive value” relates to a predictive value of tests, which is the probability of a target condition given by the result of a test. This is often applied in medical testing. In cases where binary classification can be applied to the test results, such “yes” versus “no”, (for example, test target (such as a substance, symptom, or sign) being present versus absent, or either a positive or negative test), then each of the two outcomes has a separate predictive value. For example, for positive or negative test, the predictive values are termed positive predictive value or negative predictive value, respectively. In cases where the test result is of a continuous value, the predictive value generally changes continuously along with the value. For example, for a pregnancy test that displays the urine concentration of human chorionic gonadotropin (hCG), the predictive value increases with increasing human chorionic gonadotropin (hCG) value. A conversion of continuous values into binary values can be performed, such as designating a pregnancy test as “positive” above a certain cut-off value, however this also confers a loss of information and generally results in less accurate predictive values.

As used herein, the term “normalised”, as used in the context of statistics, can have a range of meanings. In the simplest cases, normalisation of ratings means adjusting values measured on different scales to a notionally common scale, often prior to averaging. In more complicated cases, normalization may refer to more sophisticated adjustments where the intention is to bring the entire probability distributions of adjusted values into alignment. A different approach to normalization of probability distributions is quantile normalization, where the quantiles of the different measures are brought into alignment. In another usage in statistics, normalization refers to the creation of shifted and scaled versions of statistics, where the intention is that these normalized values allow the comparison of corresponding normalized values for different datasets in a way that eliminates the effects of certain gross influences, as in an anomaly time series. Some types of normalization involve only a rescaling, to arrive at values relative to some size variable. In the present disclosure, the level of polyamine(s) is normalised. In another example, the normalisation is done with creatinine.

As used herein the term “ROC”, “receiver operating characteristic curve”, or “ROC curve”, is a graphical plot that illustrates the diagnostic ability of a binary classifier system as its discrimination threshold is varied. The method was originally developed for operators of military radar receivers, which is why it is so named.

The ROC curve is created by plotting the true positive rate (TPR) against the false positive rate (FPR) at various threshold settings. The true-positive rate is also known as sensitivity, recall or probability of detection in machine learning. The false-positive rate is also known as probability of false alarm and can be calculated as (1—specificity). It can also be thought of as a plot of the power as a function of the Type I Error of the decision rule (when the performance is calculated from just a sample of the population, it can be thought of as estimators of these quantities). The ROC curve is thus the sensitivity or recall as a function of fall-out. In general, if the probability distributions for both detection and false alarm are known, the ROC curve can be generated by plotting the cumulative distribution function (area under the probability distribution from −∞ to the discrimination threshold) of the detection probability in the y-axis versus the cumulative distribution function of the false-alarm probability on the x-axis.

In conjunction with the receiver operating characteristic curve, the term “AUC” refers to the area under the curve (often referred to as simply the AUC), which is an area equal to the probability that a classifier will rank a randomly chosen positive instance higher than a randomly chosen negative one (assuming ‘positive’ ranks higher than ‘negative’). In other words, the closer the value to 1 (AUC values range from 0 to 1), the higher the probably that the outcome of the chosen test will be correct. It is of note that an AUC of 0.5 indicates that the outcome will be uninformative.

As used herein, the term “logistic regression”, also known as logit regression, refers to a statistical model used to model the probability of a certain class or event existing, such as, but not limited to pass/fail, win/lose, alive/dead or healthy/sick. This can be extended to model several classes of events, such as determining whether an image contains a cat, dog, lion, for example. Each object being detected in the image would be assigned a probability between 0 and 1, with a sum of one.

Logistic regression is a statistical model that, in its basic form uses, a logistic function to model a binary dependent variable, although many more complex extensions exist. In regression analysis, logistic regression (or logit regression) is estimating the parameters of a logistic model (a form of binary regression).

Mathematically, a binary logistic model has a dependent variable with two possible values, such as pass/fail, each outcome of which is represented by an indicator variable, where the two values are labelled “0” and “1”. In the logistic model, the log-odds (the logarithm of the odds) for the value labelled “1” is a linear combination of one or more independent variables (also referred to as “predictors”); the independent variables can each be a binary variable (two classes, coded by an indicator variable) or a continuous variable (any real value). The corresponding probability of the value labelled “1” can vary between 0 (certainly the value “0”) and 1 (certainly the value “1”), hence the labelling. The function that converts log-odds to probability is the logistic function, hence the name. The unit of measurement for the log-odds scale is called a logit, from logistic unit, hence the alternative names. Analogous models with a different sigmoid function instead of a logistic function can also be used, such as, but not limited to, the so-called probit model. The defining characteristic of the logistic model is that increasing one of the independent variables multiplicatively scales the odds of the given outcome at a constant rate, with each independent variable having its own parameter; for a binary dependent variable this generalizes the odds ratio.

In a binary logistic regression model, the dependent variable has two levels (categorical). Outputs with more than two values are modelled by multinomial logistic regression and, if the multiple categories are ordered, by ordinal logistic regression (for example the proportional odds ordinal logistic model). The logistic regression model itself simply models probability of output in terms of input and does not perform statistical classification (and it is therefore not considered to be a classifier). However, this does not preclude the logistic regression model from being able to be used to make a classifier. This can be done, for example, by choosing a cut-off value and classifying inputs with probability greater than the cut-off as one class, and inputs with probability lower than the cut-off as the other class. This is a common way to make a binary classifier.

As used herein, the terms “increase” and “decrease” refer to the relative alteration of a chosen trait or characteristic in a subset of a population in comparison to the same trait or characteristic as present in the whole population. An increase thus indicates a change on a positive scale, whereas a decrease indicates a change on a negative scale. The term “change”, as used herein, also refers to the difference between a chosen trait or characteristic of an isolated population subset in comparison to the same trait or characteristic in the population as a whole. However, this term is without valuation of the difference seen.

As used herein, the term “about” in the context of concentration of a substance, size of a substance, length of time, or other stated values means +/−5% of the stated value, or +/−4% of the stated value, or +/−3% of the stated value, or +/−2% of the stated value, or +/−1% of the stated value, or +/−0.5% of the stated value.

As used herein, the term “monitoring” refers to the (medical) observation of a disease, condition, or one or several medical parameters over time. These parameters may or may not be related to a specific disease. This monitoring can be performed by continuously measuring certain parameters by using a medical monitor (for example, by continuously measuring vital signs by a bedside monitor), and/or by repeatedly performing medical tests (such as, for example, blood glucose monitoring with a glucose meter in people with diabetes mellitus).

As used herein, the term “surgery” refers to a procedure that uses operative manual and instrumental techniques on a subject to investigate and/or treat a pathological condition such as a disease or injury. Surgery can also be performed for cosmetic reasons.

As used herein the term “castration” refers to a procedure which results in the removal or loss of use of the testicles. This procedure is also referred to as an orchiectomy or orchidectomy. This procedure can be performed surgically, chemically, or using any other method, resulting in the loss of the testicles, that is, the male gonad. Surgical castration is bilateral orchiectomy (excision of both testicles), while a chemical castration (also referred to as medical castration) uses drugs to deactivate the testes. Castration causes sterilization (preventing the castrated person or animal from reproducing) and greatly reduces the production of certain hormones, such as testosterone.

### DETAILED DESCRIPTION OF THE PRESENT INVENTION

Prostate-specific antigen (PSA) has been commonly used as a tool for early prostate cancer (PCa) detection. The 16-year update of the European randomized study of screening for prostate cancer showed that screening with prostate-specific antigen could reduce prostate cancer mortality, and one prostate cancer death could be saved by screening 570 men and treating 18 men. However, using prostate-specific antigen as the main screening tool had resulted in a lot of unnecessary biopsies, diagnosis, and treatment of indolent (that is to say, slow growing, low grade) prostate cancers. Current methods allow the use of a plethora of diagnostics tools utilizing blood (such as, but not limited to, for example, Prostate Health Index, 4-kallikrein panel), urine (for example, but not limited to, post-prostate massage samples in PCA3 and SelectMDx), or imaging (for example, but not limited to, multi-parametric MRI of the prostate) to significantly improve diagnostic accuracy of prostate cancer and reduce unnecessary biopsies.

Polyamines are involved in growth and proliferation of prostatic glandular epithelial cells and, for example, spermidine and putrescine, are present in high levels in human prostate tissue. Polyamines, for example, putrescine and spermidine, were shown to be increased in proliferating prostate cancer cells. Spermine, for example, has been shown to be involved in secretory function of prostate epithelial cells, and is normally concentrated in benign prostate tissue with large luminal volumes. Prostate cancer, with changes in cellular architecture and reduced luminal volumes, especially in cases with poor cellular differentiation, was shown to have lower levels of spermine in cancer tissue. It is noted that the comparison here was made between well differentiated prostate cancer and poorly differentiated prostate cancer, whereby it was shown that more poorly differentiated prostate cancer has lower levels of spermine compared to the more well-differentiated prostate cancer.

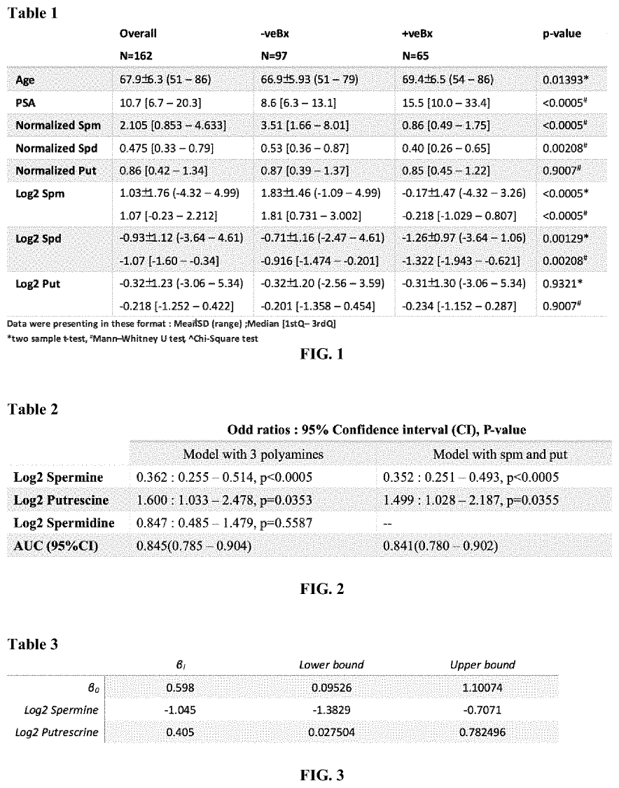

As disclosed herein, urine samples of 162 patients with a prostate-specific antigen concentration of more than 4 ng/ml were collected. The patients' age for these urine samples ranged from the 51 years to 86 years of age. The prostate-specific antigen concentration ranged from 4.2 ng/ml to 299 ng/ml. Descriptive statistics and comparisons between +veBx (positive prostate biopsy result, meaning that cancer cells were found in the biopsy samples) vs −veBx (negative prostate biopsy result, meaning that no cancer cells were found) groups are summarized in Table 1 (FIG. 1). The difference of age, prostate-specific antigen (PSA), normalized spermine (spm) and normalized spermidine (spd) were observed between the +veBx vs −veBx groups. The area under the curve (AUC) values of spermine, spermidine and putrescine are 0.83, 0.64 and 0.51, respectively, (see FIGS. 6 to 8). The three studied urinary polyamines were put into a backward logistic regression analysis in order to predict biopsy results. Log 2Spm and Log 2Put together also shows the statistical usefulness, Table 2 (FIG. 2) listed the odd ratios calculated from the logistic regressions.

The predicted probability of positive biopsy results with Log 2Spm and Log 2Put together at 95% sensitivity, 44% specificity, threshold value is 0.180; at 90% sensitivity, 49% specificity, threshold value is 0.196; at the best point—80% sensitivity, 72% specificity, threshold value is 0.353. The statistical model disclosed herein uses two factors, log 2 transformed normalised spermine and log 2 transformed normalised putrescine, to calculate and predict a positive biopsy result.

Pr(+veBx|Log 2Spm, Log 2Put)=1/1+exp−(0.598−1.045*Log 2Spm+0.405*Log 2Put) Formula

The above formula is a standard logistic regression formula, whereby the coefficients were generated with logistic regression, using data obtained from 162 subjects. Specifically, the coefficients disclosed in the formulae disclosed herein were generated using statistics programming language with logistic regression function. All data collected was imported into the programming environment and the coefficients were generated with the logistic regression model.

Two different models have been derived to calculate the combined score of positive biopsy results one with three parameters (age, prostate-specific antigen (PSA), and log 2spermine (log 2spm)), and the other model with age, prostate-specific antigen, log 2spermine (log 2spm) and log 2putrescine (log 2put). Other parameters can also be added into the prediction model.

Pr(y=1|xᵢ)=1/1+exp−(β₀+βᵢxᵢ) Logistic regression formula

(see Table 3 (FIG. 3) for the coefficients and their range for the main model)

With regard to the formula shown above, βᵢ represents regression coefficients, β₀ represents the intercept, and xᵢ are the values of the matching independent variables. The result (Pr(y=1|xᵢ)) is the probability for an observation with the given pattern of values of the independent variables to have the event. These Pr(y=1|xᵢ) are the scores that are used to build the receiver operating characteristic (ROC) curve.

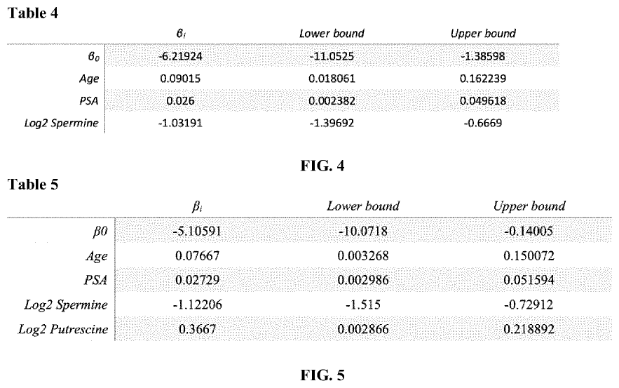

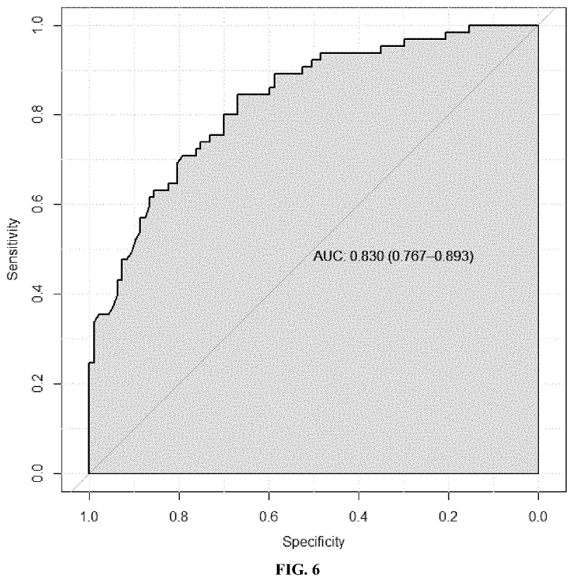

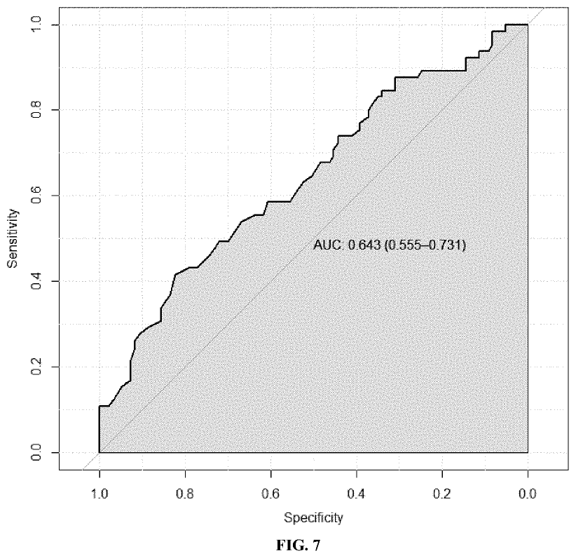

Model 1. Predicted outcome as a function of Age, Psa, Log 2Spm (see Table 4 (FIG. 4)).

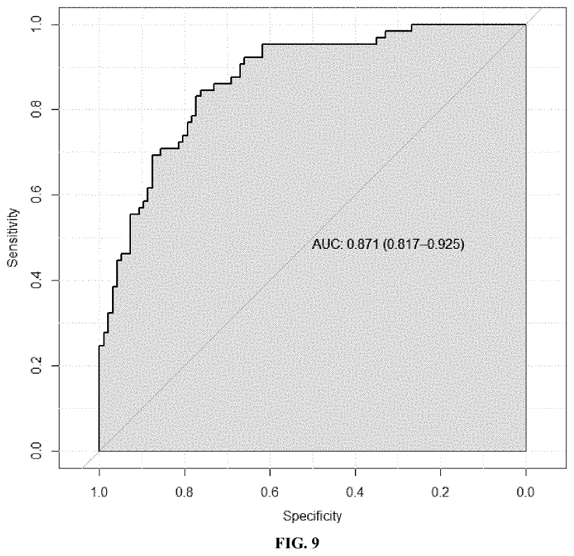

AUC=0.871, 95% CI: 0.817-0.925 (see FIG. 9).

Pr(+veBx|Age,Psa, Log 2Spm)=1/1+exp−(−6.219+0.090*Age+0.026*PSA−1.032*Log 2Spm) Formula

The formula is a standard logistic regression formula, the coefficients were generated using the 162-patient data with logistic regression. AUC is the area under the ROC curve plotted with different threshold of the predicted probability of the model. 95% CI means 95% confidence interval. As “95% CI” appear after “AUC”, this indicates that the 95% confidence interval is of the AUC value.

The cut-off values of the combined scores with model 1: at 95% sensitivity, 62% specificity, threshold value is 0.222; at 90% sensitivity, 67% specificity, threshold value is 0.276; at the best point—85% sensitivity, 76% specificity, threshold value is 0.348 (see Table 4 (FIG. 4)).

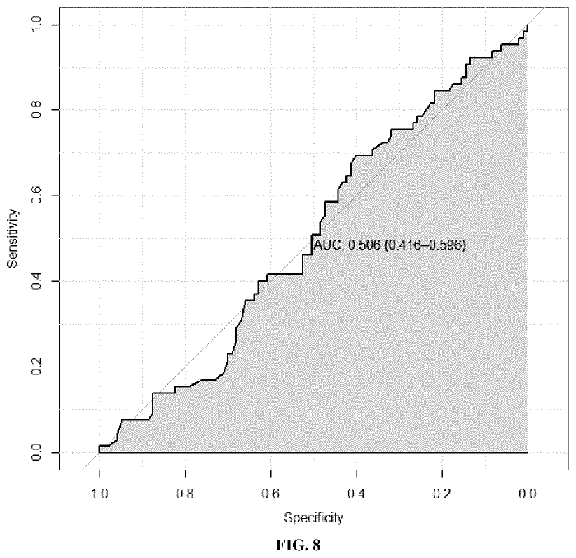

Model 2. Predicted outcome as a function of Age, Psa and Log 2Spm and log 2Put (See Table 5 (FIG. 5)).

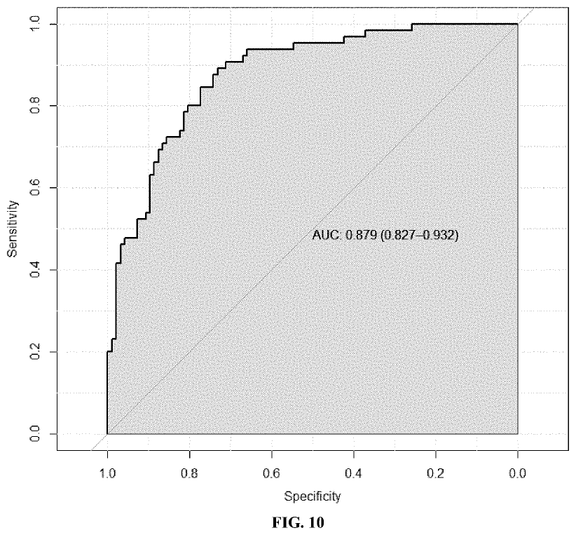

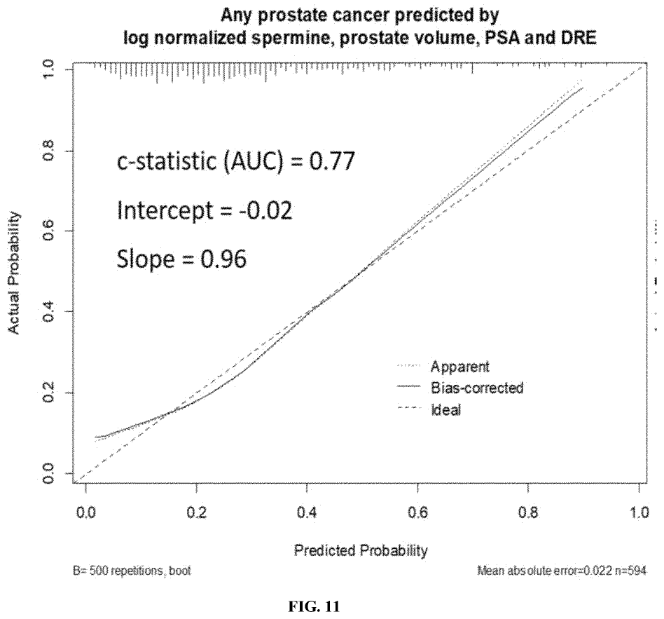

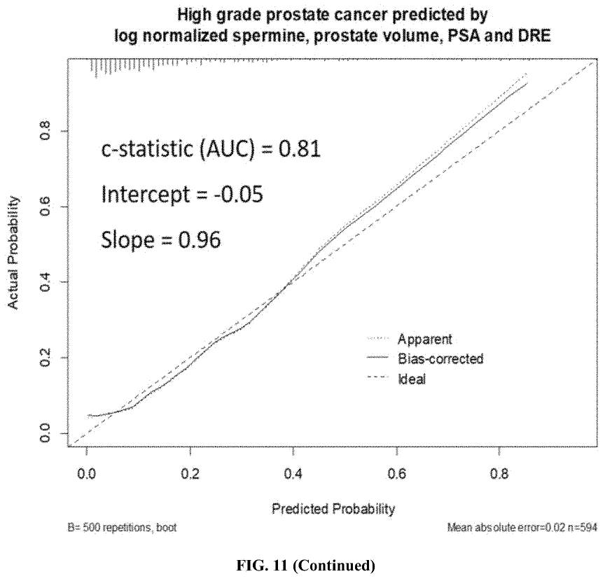

AUC=0.879 (0.827-0.932) (see FIG. 10).

Pr(PC|Age,Psa, Log 2Spm, Log 2Put)=1/1+exp−(−5.106+00.077*Age+00.027*PSA−1.122*Log 2Spm+0.367*Log 2Put) Formula

The formula is a standard logistic regression formula, the coefficients were generated using the 162-patient data with logistic regression. AUC is the area under the ROC curve plotted with different threshold of the predicted probability of the model. 95% CI means 95% confidence interval. As “95% CI” appear after “AUC”, this indicates that the 95% confidence interval is of the AUC value.

The cut-off values of the combined scores with model 2: at 95% sensitivity, 55% specificity, threshold value is 0.173; at 90% sensitivity, 71% specificity, threshold value is 0.276; at the best point—89% sensitivity, 73% specificity, threshold value is 0.309.

Urine polyamines, such as for example, spermidine, putrescine, and spermine had been reported to be associated with various types of cancers. Studies have described higher proportion of elevated 24-hour urine spermidine concentration in prostate cancer compared with non-cancer controls. Elevated 24-hour urine putrescine, but not spermidine, has been previously described in 30 prostate cancer patients, while spermine was shown not to be detectable in most urine samples using chromatographic analysis. The potential role of 24-hour urine diamine, spermidine, and spermine in predicting prostate cancer had been previously reported in a small cohort of 17 men. The same group subsequently reported on a 24-hour urine polyamine enzyme test kit for urological cancers, but the predictive ability for prostate cancer was limited. However, it is noted that none of the prior art provided any conclusion as to the usefulness of urine polyamines in prostate cancer diagnosis. The data disclosed in the present application (based on 162 subjects) shows that spermine has an AUC of >0.8 in prostate cancer detection. In further experiments (600 subjects), the use of spermine in addition with clinical data has been used as a prediction model for prostate cancer risk prediction.

In one example, polyamines, for example, urine polyamine, were investigated for their applicability in the context of prostate cancer. In one pilot study, it was found that urine spermine without prior prostatic massage correlated with prostate cancer.

Thus, disclosed herein is data obtained about the applicability of urine polyamines and a risk score (for example, a Spermine Risk Score) in predicting the presence of prostate cancer in a subject. In the methods disclosed herein, this study was performed on a consecutive cohort of men at risk of prostate cancer.

The study disclosed herein comprised of 905 men, who had undergone prostate biopsies, and who had had pre-biopsy urine sent for spermine analysis. The median prostate-specific antigen was 9.6 ng/ml (interquartile range (IQR) 6.4-16.5 ng/ml). Most patients received systematic prostate biopsies of a median of 14 cores (IQR 10-24). In the whole cohort, prostate cancer (PCa) and high-grade prostate cancer (HGPCa) were diagnosed in 44.5% (403/905) and 25.9% (234/905) of men, respectively. A lower urine spermine level was significantly associated with higher risks of prostate cancer (PCa) and high-grade prostate cancer (HGPCa) (chi-square test, p<0.001 for PCa and HGPCa).

Thus, in one example, the subject had been determined to have a prostate-specific antigen (PSA) concentration of at least 4 ng/ml prior to preforming the method. In another example, the prostate-specific antigen (PSA) concentration is at least 4 ng/ml, at least 5 ng/ml, at least 6 ng/ml, at least 7 ng/ml, at least 8 ng/ml, at least 9 ng/ml, at least 10 ng/ml, at least 11 ng/ml, at least 12 ng/ml, at least 13 ng/ml, at least 14 ng/ml, at least 15 ng/ml, at least 16 ng/ml, at least 17 ng/ml, at least 18 ng/ml, or at least 19 ng/ml; or about 4 ng/ml, about 5 ng/ml, about 6 ng/ml, about 7 ng/ml, about 8 ng/ml, about 9 ng/ml, about 10 ng/ml, about 11 ng/ml, about 12 ng/ml, about 13 ng/ml, about 14 ng/ml, about 15 ng/ml, about 16 ng/ml, about 17 ng/ml, about 18 ng/ml, about 19 ng/ml, about 20 ng/ml, or about 21 ng/ml. In another example, the prostate-specific antigen (PSA) concentration is between 3 ng/ml to 22 ng/ml, between 3 ng/ml to 5 ng/ml, between 5 ng/ml to 7 ng/ml, between 3 ng/ml to 9 ng/ml, between 4 ng/ml to 13 ng/ml, between 6 ng/ml to 18 ng/ml, between 7 ng/ml to 19 ng/ml, between 8 ng/ml to 20 ng/ml, between 9 ng/ml to 18 ng/ml or between 10 ng/ml to 20 ng/ml.

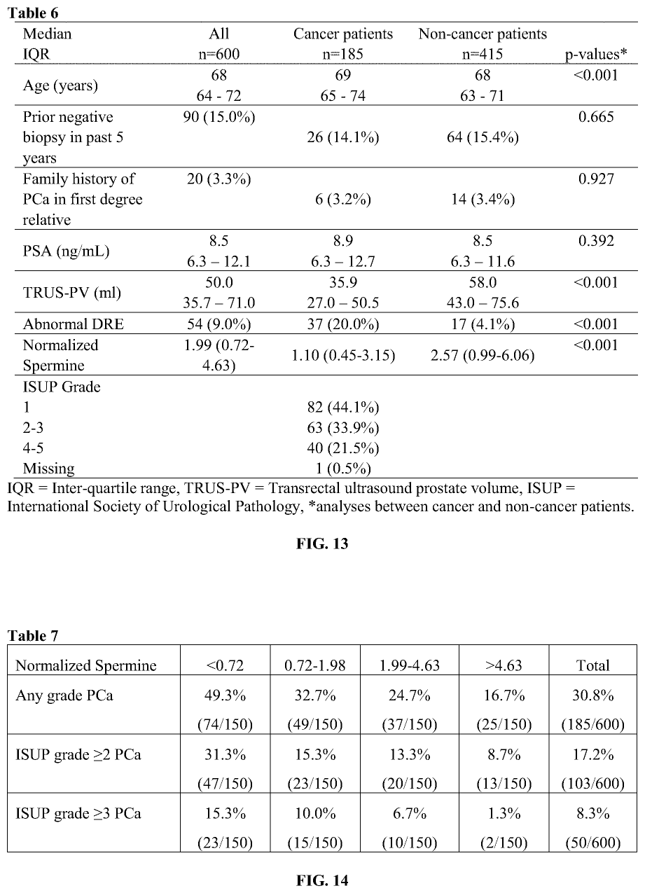

In a recent study, among the 905 men, 305 men showing a prostate-specific antigen level of higher than 20 ng/ml were excluded from analysis. The resulting 600 men with a prostate-specific antigen level of within the range of 4 to 20 ng/ml, and without prior diagnosis of prostate cancer, were included in the main analysis group. The baseline characteristics of these 600 men are shown in Table 6 (FIG. 13). Only 20.8% (125/600) men had undergone a pre-biopsy MRI, and only 13.7% (82/600) had undergone an MRI-guided biopsy.

Prostate cancer (PCa) and high-grade prostate cancer (HGPCa) were diagnosed in 30.8% (185/600) and 17.2% (103/600) of men, respectively, and the present of prostate cancer was significantly associated with lower urine spermine levels (chi-square test, p<0.001) (Table 2). Between the highest and lowest quartiles of spermine results, a threefold increase in prostate cancer risk (49.3% vs. 16.7%), a 3.5-fold increase in ISUP GG ≥2 PCa risk (31.3% vs. 8.7%), and 11-fold increase in ISUP GG ≥3 PCa risk (15.3% vs. 1.3%) were observed. Prior negative biopsy or family history of prostate cancer was not shown to be associated with prostate cancer or high-grade prostate cancer based on biopsy results.

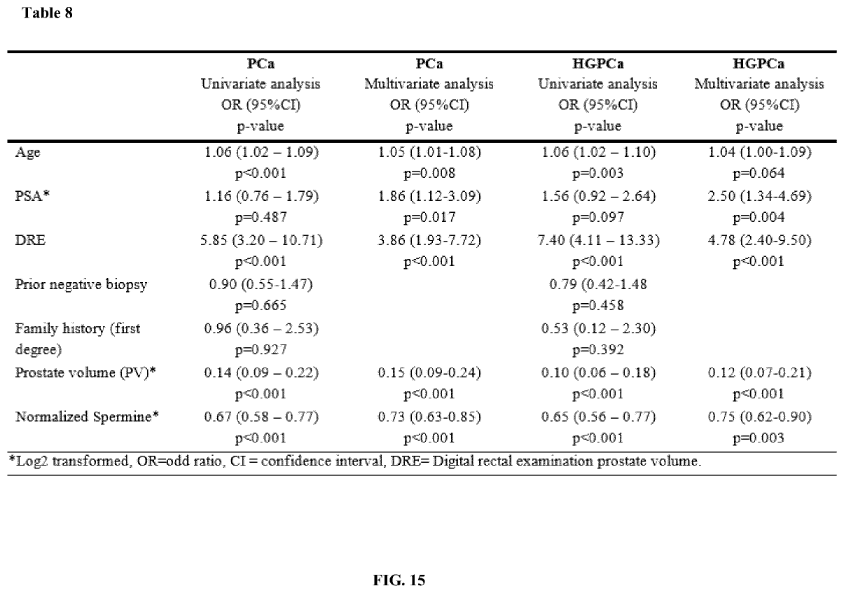

Age, digital rectal examination (DRE), and natural logarithm values of spermine, prostate-specific antigen (PSA) and prostate volume (PV) are analysed using univariate and multivariate analyses. Univariate analyses showed that age, prostate volume, digital rectal exam, and spermine were all significant predictors for prostate cancer and high-grade prostate cancer (Table 8, FIG. 15). Multivariate analyses showed that age, prostate-specific antigen (PSA), prostate volume (PV), digital rectal examination (DRE), and spermine are independent predictors for prostate cancer, while prostate-specific antigen (PSA), prostate volume (PV), digital rectal examination (DRE), and spermine are independent predictors for high-grade prostate cancer (Table 8, FIG. 15).

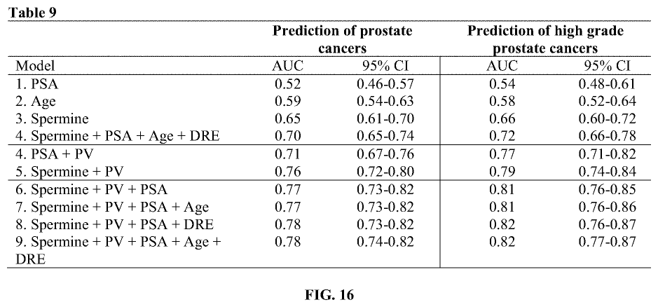

For prostate cancer (PCa) and high-grade prostate cancer, area under the curve (AUC) in receiver operating characteristic (ROC) analyses of the above factors and combination of factors are listed in Table 9 (FIG. 16).

Various models including the variables and polyamines disclosed herein are shown. In one example, the method disclosed herein comprises at least one polyamine and at least one variable.

In one example, the model comprising spermine, prostate volume, prostate-specific antigen, and digital rectal examination achieved the highest AUC in both prostate cancer (0.78) and high-grade prostate cancer (0.82).

A four-factor Spermine Risk Score for high-grade prostate cancer (International Society of Urological Pathology (ISUP) grade ≥2) was obtained based on AUC values, Akaike information criterion (AIC), and Bayesian information criterion (BIC). This risk score is calculated by using logistic regression, with coefficients generated using the 600-patient data. Digital rectal examination (DRE) results were coded either as 1 (for positive DRE results) or 0 (for negative DRE results).

Thus, in one example, the score value is calculated using i) area under the curve (AUC) of receiver operating characteristics (ROC), logistic regression, Akaike information criterion (AIC), and Bayesian information criterion (BIC); ii) p-value based on a test selected from the group consisting of student's t-test, Mann-Whitney U test, Chi-square test, two-sided t-test; and/or iii) a classification algorithm such as, but not limited to, support vector machine algorithm, logistic regression algorithm, multinomial logistic regression algorithm, Fisher's linear discriminant algorithm, quadratic classifier algorithm, perceptron algorithm, k-nearest neighbour's algorithm, artificial neural network algorithm, random forests algorithm, decision tree algorithm, naive Bayes algorithm, adaptive Bayes network algorithm, and ensemble learning method combining multiple learning algorithms.

In one example, the classification algorithm is pre-trained using the level of the one or more polyamines of the control, and at least one variable selected from the group consisting of age, prostate volume (PV), prostate-specific antigen (PSA), digital rectal examination (DRE), and combinations thereof, of the control. In another example, the classification algorithm compares the level of the one or more polyamines present in the sample obtained from the subject and at least one variable, with that of the control, and returns a mathematical score that identifies the likelihood of the subject to belonging to the control or not.

Thus, in one example, the method of determining the risk of developing prostate cancer in a subject or determining whether a subject suffers from prostate cancer comprising measuring the level of one or more polyamines in a fluid sample obtained from the subject, measuring at a variable selected from the group consisting of age, prostate volume (PV), prostate-specific antigen (PSA), digital rectal examination (DRE), and combinations thereof; comparing the level of the one of more polyamines and the at least one variable to a control.

In another example, there is disclosed a method of determining the risk of developing prostate cancer in a subject or determining whether a subject suffers from prostate cancer, the method comprising measuring the level of one or more polyamines in a fluid sample obtained from the subject, measuring a variable, wherein the variable is, but is not limited to, age, prostate volume (PV), prostate-specific antigen (PSA), digital rectal examination (DRE), and combinations thereof; obtaining a score value based on the level of the one or more polyamines measured in step c. and the at least one variable measured in step d. to predict the likelihood of the subject developing or having prostate cancer; wherein an increase in score value indicates that the subject is at risk of developing, or suffers from, prostate cancer.

In one example, the variable is, but not limited to, the following: age, prostate volume (PV), prostate-specific antigen (PSA), digital rectal examination (DRE), and combinations thereof. In another example, the variable is a combination of any of the variables disclosed therein. In another example, the variable is a combination of, but not limited to, age and prostate volume, age and prostate-specific antigen, age and digital rectal examination, prostate volume and prostate-specific antigen, prostate volume and digital rectal examination, and prostate-specific antigen and digital rectal examination. In yet another example, the variable is a combination of, but not limited to age, prostate volume, and prostate-specific antigen; age, prostate volume, and digital rectal examination; age, prostate-specific antigen and digital rectal examination; and prostate volume, prostate-specific antigen, and digital rectal examination. In yet another example, the variable is a combination of age, prostate volume, prostate-specific antigen, and digital rectal examination.

Thus, in one example, there is disclosed a method of determining the risk of developing prostate cancer in a subject or determining whether a subject suffers from prostate cancer, the method comprising measuring the level of one or more polyamines in a fluid sample obtained from the subject, measuring at a variable selected from the group consisting of age, prostate volume (PV), prostate-specific antigen (PSA), digital rectal examination (DRE), and combinations thereof; comparing the level of the one of more polyamines and the at least one variable to a control; wherein a decrease or increase in the level of the one or more polyamines compared to the control indicates that the subject is at risk of developing, or suffers from, prostate cancer; wherein a decrease in prostate volume and/or an increase prostate-specific antigen (PSA) compared to the control indicates that the subject is at risk of developing, or suffers from, prostate cancer; wherein a positive digital rectal examination result indicates that the subject is at risk of developing, or suffers from, prostate cancer; wherein the one or more polyamines is selected from the group consisting of spermine, spermidine and putrescine.

In another example, there is disclosed a method of determining the risk of developing prostate cancer in a subject or determining whether a subject suffers from prostate cancer, the method comprising measuring the level of one or more polyamines in a fluid sample obtained from the subject, measuring a variable selected from the group consisting of age, prostate volume (PV), prostate-specific antigen (PSA), digital rectal examination (DRE), and combinations thereof; obtaining a score value based on the level of the one or more polyamines measured in a previous step and the at least one variable measured in a previous step to predict the likelihood of the subject developing or having prostate cancer; wherein an increase in score value indicates that the subject is at an increased risk of developing, or suffers from, prostate cancer.

A person skilled in the art will appreciate that the score value obtained using the methods disclosed herein and the risk of said subject developing, or suffering from, prostate cancer is in a positive correlation to each other. That is to say, for example, an increase in risk of a subject developing, or suffering from, prostate cancer results in an increase in the score obtained for said subject. The same also applied in the opposite scenario. That is to say that a decrease in risk of a subject developing, or suffering from, prostate cancer results in a decrease in the score obtained for said subject.

In one example, the methods disclosed herein refer to one or more polyamines. In another example, the polyamine is, but is not limited to, spermine, spermidine, or putrescine. A polyamine can also be a combination of polyamines (one or more polyamines) as disclosed herein. In one example, the polyamine is a combination of, but not limited to, spermine and spermidine, spermine and putrescine, and spermidine and putrescine. In another example, the polyamine is spermine. In yet another example, the polyamine is a combination of putrescine and spermine. In yet another example, the polyamine is a combination of putrescine, spermidine, and spermine.

It is noted that an increase in prostate-specific antigen, spermidine and/or putrescine indicates an increased risk of developing, or the presence of, prostate cancer. Furthermore, a decrease in prostate volume and/or spermine also indicates an increased risk of developing, or the presence of, prostate cancer.

In one example, the method uses any one of the polyamines disclosed herein and a variable as disclosed herein. Thus, in one example, the polyamine is spermidine, and the variable as disclosed herein. In another example, the polyamine is putrescine, and the variable as disclosed herein. In another example, the polyamine is spermine, and the variable as disclosed herein. In yet another example, the polyamine is spermine, and the variable is prostate volume. In yet another example, the polyamine is spermine, and the variable is a combination of prostate volume and prostate-specific antigen. In a further example, the polyamine is spermine, and the variable is a combination of prostate volume, prostate-specific antigen and age. In one example, the polyamine is spermine, and the variable is a combination of prostate volume, prostate-specific antigen and digital rectal examination. In a further example, the polyamine is spermine, and the variable is a combination of prostate volume, prostate-specific antigen, age, and digital rectal examination. In yet another example, the polyamine is spermine, and the variable is a combination of prostate-specific antigen, age and digital rectal examination.

In one example, a decrease in prostate volume and/or an increase in prostate-specific antigen (PSA) compared to the control indicates that the subject is at risk of developing, or suffers from, prostate cancer. By the same line, an increase in prostate volume and/or a decrease in prostate-specific antigen (PSA) compared to the control indicates that the subject is not at risk of developing, or suffers from, prostate cancer.

In one example, the digital rectal examination returns a positive result. In another example, the digital rectal examination returns a negative result. In another example, a positive digital rectal examination result indicates that the subject is at risk of developing, or suffers from, prostate cancer. By the same line, a negative digital rectal examination result indicates that the subject is not at risk of developing, or suffers from, prostate cancer. As defined herein, the results of a digital rectal examination are defined as either “0” corresponding to a negative examination result, or “1”, corresponding to a positive examination result, respectively.

As used herein, the term “control”, “control group”, “negative control” or “control” when used in the context of sample analysis, refers to the use of samples obtained from diseases-free or healthy subjects, whereby these samples are then treated in the same manner as other sample, with the difference that the control samples are treated with, for example, buffers that do not contain the active compound or molecule in question. The comparison of the concentration of one or more targets (for example when comparing absolute concentrations or relative expression levels of a target), or the determination of the presence or absence of one or more targets as disclosed herein (for example, one or more proteins, oligomers or oligonucleotides) is determined based on the comparison of the levels determined in a sample obtained from a diseased subject and a sample obtained from a disease-free (or healthy) subject. In other words, a comparison of a target is based on the comparison of the level of one or more targets determined in the diseased subject and the level of the same one or more targets determined in a control group or control individual. In the present disclosure, the control sample is obtained from an individual that is disease-free. That is to say that the individual, from which the control sample is obtained, is free of the disease for which the test is undertaken. Usually, the term disease-free implies that the subject is healthy. Thus, in one example, the control is a cancer-free subject. In another example, the control is a subject with a prostate cancer of ISUP<2. In one example, when calculating the risk score of high-grade prostate cancer, the control is subject with ISUP<2 prostate cancers. This means that, in this example, the control includes subjects suffering from low-grade cancer. In another example, when calculating the (risk) score disclosed herein, the control is cancer-free subjects. It is further noted that this (risk) score, as disclosed herein, can be applied to any grade of prostate cancer.

The term “sample” includes, but is not limited to, any quantity of a substance from a living thing or formerly living thing. Such living things include, but are not limited to, humans, mice, monkeys, rats, rabbits, and other animals. Such substances or samples are, but are not limited to, cellular and non-cellular components of amniotic fluid, breast milk, bronchial lavage, cerebrospinal fluid, colostrum, interstitial fluid, peritoneal fluids, pleural fluid, saliva, seminal fluid, urine, tears, whole blood, plasma, serum plasma, and serum. In one example, the method is performed on a fluid sample. In another example, the fluid sample is, but is not limited to, urine, whole blood, plasma, serum plasma, and serum. In yet another example, the fluid sample is urine.

For prediction of ISUP grade ≥3 prostate cancer, AUC of the model including spermine, prostate volume, prostate-specific antigen, age, and digital rectal examination achieved the AUC of 0.85 ((Table 9, FIG. 16).

When urine spermine is used alone or in combination with other factors, it was shown that a proportion of unnecessary biopsies can be reduced at different thresholds. Using 90% sensitivity (in other words, a scenario whereby 10% cases are missed, or are false), for high-grade prostate cancer, using a spermine cut-off of 5.35 could reduce biopsies by 22% (132/600) at a negative predictive value (NPV) of 92.4%. At 90% sensitivity for high-grade prostate cancer, the Spermine Risk Score including spermine, prostate volume, prostate-specific antigen and digital rectal examination could reduce biopsies by 36.7% (218/594) with negative predictive value of 95.4% (208/218) (at a Spermine Risk Score cut-off of 7) and avoid (false) diagnosis in 24.4% (20/82) of ISUP grade 1 prostate cancers. The positive predictive value (at Spermine Risk Score cut-off of 7) was 24.5% (92/376). The risk of having high-grade prostate cancer with four-factor Spermine Risk Score was 24.5% (score ≥7) and 4.6% (score <7) (chi-square test, p=<0.001).

At 95% sensitivity for high-grade prostate cancer (Spermine Risk Score cut-off at 4.9), the Spermine Risk Score could reduce biopsies by 22.9% (136/594) with NPV of 96.3% (131/136) and avoided (false) diagnosis in 12.2% (10/82) ISPU GG 1 prostate cancers.

At 90% sensitivity for ISUP GG ≥3 prostate cancers, the spermine model including spermine, prostate volume, prostate-specific antigen, age, and digital rectal examination could reduce biopsies by 49.3% (294/596) with a negative predictive value of 97.3% (286/294) at cut-off value of 5. The risk of ISUP GG ≥3 prostate cancers with this spermine model was 13.9% (score ≥5) and 2.7% (score <5; chi-square test, p≤0.001).

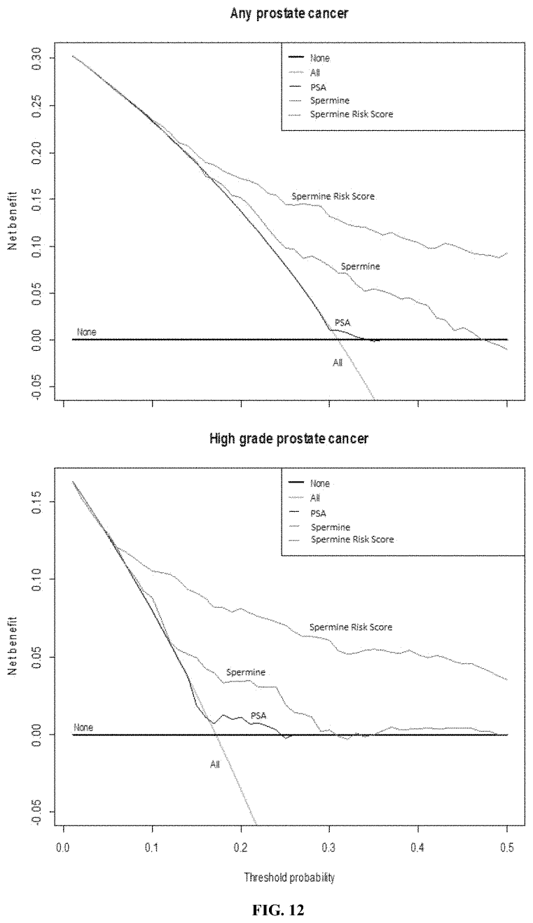

Decision curve analyses (DCA; FIG. 12) showed that Spermine Risk Score has a net clinical benefit over normalized spermine, prostate-specific antigen density, or prostate-specific antigen alone in prediction of both prostate cancer and high-grade prostate cancer (FIG. 11). For high-grade prostate cancer, the clinical benefit was observed from any threshold probability above 5%. Internal validation of Spermine Risk Score for high-grade prostate cancer using the Bootstrapping method resulted in good discrimination and calibration with AUC of 0.81, slope of 0.96, and intercept of −0.05 (FIG. 11).

The data disclosed herein was obtained to ascertain the role of polyamines for example, but not limited to, urine spermine in the detection of prostate cancer. This data had been gathered from men who had shown an elevated prostate-specific antigen concentration (>4 ng/mL) and who had undergone prostate biopsy. A lower level of urine normalized spermine was shown herein to be associated with higher risk of prostate cancer and high-grade prostate cancer. By dividing normalised spermine into different reference ranges by quartiles, it was shown that decreasing levels of urine spermine was associated with a progressive increase in the risk of any grade prostate cancer, ISUP GG ≥2 cancers, and ISUP GG ≥3 cancers (Table 9, FIG. 16). This is in line with the presence of a lower level of spermine in malignant prostatic tissues or high-grade prostate cancer.

As shown herein, prostate cancer and high-grade prostate cancer were diagnosed in 30.8% (185/600) and 17.2% (103/600) of men, respectively, in the current cohort of 600 Chinese men with prostate-specific antigen concentrations of between 4 to 20 ng/ml. This cancer detection rate is similar to reported rates in Asian men with similar PSA range, which is well-known to be lower than that in Caucasian population

It is shown herein that the performance of urine spermine for use in detecting prostate cancer can be improved by a multivariable risk model including, for example, prostate volume, prostate-specific antigen, and digital rectal examination findings/results. The AUC value of this multivariable risk model (i.e., a Spermine Risk Score) for prostate cancer and high-grade prostate cancer was found to be higher for detection based on prostate-specific antigen density or spermine alone, thereby preventing unnecessary biopsies by up to 36.7% (at 90% sensitivity for high-grade prostate cancer) with a negative prediction value of 95.4%. Decision curve analysis showed net clinical benefit of the Spermine Risk Score compared with other parameters for both prostate cancer and high-grade prostate cancer.

Thus, in one example, an increase in score value indicates that the subject is at risk of developing, or suffers from, prostate cancer. That is to say that a decrease in score value indicates that the subject is at a lower risk of developing, or suffers from, prostate cancer.

Only 125 out of 600 men had had a pre-biopsy prostate MRI performed. Thus, the development of Spermine Risk Score did not include PI-RADS score. When the ROC analysis was performed for these 125 men with pre-biopsy MRIs, the Spermine Risk Score performed better than other predictors: the AUC in HGPCa prediction was 0.79, 0.74, 0.64, and 0.52 for Spermine Risk Score, prostate-specific antigen density, Spermine, and prostate-specific antigen.

The effect of the Spermine Risk Score over other parameters was also seen in either transrectal or transperineal biopsies.

Although there are a number of commercially available blood and urine adjuncts to guide prostate biopsy decision in men with elevated prostate-specific antigen, urine spermine is a convenient non-invasive test without the need of another blood taking or attentive digital rectal examination before specimen collection as in the case of urine PCA3 and SelectMDx.

A small cohort of men with urine spermine after attentive digital rectal examination (three strokes per lobe) urine spermine was analysed (patients not included in this study). Numerically, it was shown that the post-attentive digital rectal examination urine spermine levels were around 3 to 4 times higher than samples without digital rectal examination. As non-digital rectal examination urine spermine already provided good differentiation between prostate cancer and non-prostate cancer, digital rectal examination was not performed before urine samples in the current cohort disclosed herein.

In the clinical application of urine spermine and the Spermine Risk Score, the urine samples are taken from subjects who have not undergone a digital rectal examination prior to urine collection. Optionally, some urine tests may require performance of a prostatic massage prior to urine collection.

In the event that a subject is determined to be suffering from prostate cancer, or has a risk of developing prostate cancer, then the method disclosed herein further comprises administered an anti-cancer drug to the subject. Alternatively, after the subject is determined to have prostate cancer, subjects will have a defined treatment plan selected from, for example, but not limited to, monitoring, surgery, surgical castration, medical castration, and/or combinations thereof. It is noted that chemotherapy and/or anti-cancer drug are not primary treatment modalities for prostate cancer.

Prostate cancer, like many other cancers, will require ongoing observation. Thus, in one example, prostate-specific antigen (PSA) monitoring is the current practice in prostate cancer treatment response. In another aspect, the method disclosed herein is use in further treatment planning and/or monitoring subject cancer status after the subject has been determined to suffer from prostate cancer.

Treatment of a subject determined to be suffering from prostate cancer, or has a risk of developing prostate cancer includes, but is not limited to, monitoring, surgery, surgical castration, medical castration, and/or combinations thereof. In some cases, a subject who has been deemed to be suffering from prostate cancer may be administered an anti-cancer drugs or a chemotherapy including, but not limited to, docetaxel, cabazitaxel, mitoxantrone, estramustine, combinations thereof and/or derivatives thereof.

Also contemplated within the scope of the present invention is a method of excluding a subject from further treatment when the risk of developing prostate cancer in a subject has been determined to be slim to none, or when a subject has been found not to be suffering from prostate cancer. This is the claimed method, but described from the opposite point in view, whereby the subject is shown not to have prostate cancer and is therefore excluded from further treatment. Thus, in one example, if the subject is identified as not being at risk of developing or suffering from prostate cancer, the subject is not subjected to further testing immediately after said identification.

Shown herein is data obtained from a study on urine spermine in consecutive men with elevated prostate-specific antigen. Urine spermine was detected by highly sensitive ultra-high performance liquid chromatography with triple quadrupole mass spectrometer (UPLC-MS/MS), compared with liquid chromatography with fluorometric detector in older studies when spermine was not well detected. Using a Spermine Risk Score approach by addition of clinical parameters (prostate-specific antigen, digital rectal examination, and prostate volume) to urine spermine was shown to further improve the prediction of high-grade prostate cancer in terms of AUC (0.82), improve decision curve analyses, and avoid biopsies. Internal validation of the Spermine Risk Score showed good calibration and discrimination.

Without being bound by theory, it is thought that the improvement in predictive performance of the risk score with the additional of prostate volume is related to higher levels of spermine being present in luminal volumes of benign enlarged prostate, which is released into the urine. In men without prostate volume information, the AUC value of the spermine model including age, prostate-specific antigen, and digital rectal examination could achieve an AUC value of 0.72 for high-grade prostate cancer. In men without digital rectal examination information, the AUC of the spermine model including age, prostate-specific antigen, and prostate volume could achieve an AUC value of 0.81 for high-grade prostate cancer.

As disclosed herein, most biopsies were done systematically, instead of MRI guided with a median of 14 cores (IQR 10-24), which may underestimate significant cancer detection. The performance of Spermine Risk Score was not compared with other prostate cancer risk calculators as it is not an externally validated cohort.

It is therefore shown that urine spermine without prostatic massage and a multivariable Spermine Risk Score can predict high-grade prostate cancer and guide biopsy decisions in men with elevated prostate-specific antigen.

Further disclosed herein is the use of one or more polyamines as disclosed herein and a variable as disclosed herein in determining the risk of developing prostate cancer in a subject or determining whether a subject suffers from prostate cancer. Also disclosed herein are one or more polyamines as disclosed herein and a variable as disclosed herein for use in determining the risk of developing prostate cancer in a subject or determining whether a subject suffers from prostate cancer.

Also contemplated in the scope of the present invention is a kit for use according to the method as described herein. In one example, the kit comprises reagents and buffers, optionally a detection system, and substances needed to carry out the method according to the present disclosure. In another example, there is disclosed one or more polyamines as disclosed herein and a variable as disclosed herein for use in a kit for determining the risk of developing prostate cancer in a subject or determining whether a subject suffers from prostate cancer.

As used in this application, the singular form “a,” “an,” and “the” include plural references unless the context clearly dictates otherwise. For example, the term “a genetic marker” includes a plurality of genetic markers, including mixtures and combinations thereof.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The invention illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and sub-generic groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples. In addition, where features or aspects of the invention are described in terms of Markush groups, those skilled in the art will recognize that the invention is also thereby described in terms of any individual member or subgroup of members of the Markush group.

### Experimental Section

### Statistical Analysis for Polyamine Analysis

The baseline characteristics were compared using T-tests (for normally distributed data), Mann Whitney U tests (for non-normally distributed data). The area under the ROC curve (AUC) for all three polyamines were determined to see how well they distinguish the disease and non-disease.

Logistic regression (LR) was used to perform outcome prediction for positive biopsy results as function of all three polyamines and as a prediction model with age, PSA level and polyamines. All urinary polyamines undergo logarithm base 2 transformation for normality and linearity to better fit the logistic regression. Discrimination ability of the models was assessed by AUC. The cut-off values of the combined scores will be determined using threshold value at 95% and 90% sensitivity; the best point (as determined by using “closest to top left” method) for each model was be determined. All statistical analyses were performed by using GraphPad Prism 6 (GraphPad Software, San Diego, Calif., USA), IBM SPSS Statistics for Windows version 25 (IBM Corp., Armonk, N.Y., USA) and R version 3.1.1 (The R foundation for statistical computing, Vienna, Austria). A two-sided p-value of <0.05 was considered statistically significant.

### Material and Methods for Human Cohort

The studying disclosed herein was performed based on data obtained from Hong Kong Chinese men. Prostate biopsies had been performed on men with elevated prostate-specific antigen (PSA) and/or abnormal digital rectal examination (DRE) without prior prostate cancer (PCa) diagnosis in two different hospitals. Institutional ethics approval was obtained before the study (CREC 2015.444). Written consent was obtained from each patient.

30 ml of urine was collected for spermine analysis before a systematic transrectal ultrasound-guided prostate biopsy via transrectal or transperineal route. No digital rectal examination (DRE) or prostatic massage was done prior to urine collection.

The urine was stored in −20° C. immediately after collection according to the standard procedures as described below. It is noted that the data generated in the present application was performed on a cohort of 162 subjects and a cohort of 600 subjects. Materials and chemicals

Methanol was obtained from TEDIA (HPLC/Spectro grade, ≥99.9%). Acetonitrile was obtained from ACS (HPLC grade, ≥99.9%). Water was purified using a MilliQ Direct Water Purification System (Millipore, USA). All standard compounds, including 1,4-Diaminobutane (Put, 99%), spermidine (Spd, ≥99.0%), spermine (Spm, ≥99.0%), 1,4-Diamino(butane-d8) dihydrochloride (98 atom % D), spermidine-(butane-d8) trihydrochloride (98 atom % D, 95% CP), spermine-(butane-d8) tetrahydrochloride (97 atom % D, 95% CP) and heptafluorobutyric acid (HFBA, ≥99.0%) were purchased from Sigma-Aldrich (Hong Kong, China) and used without further purification. Strong Anion Exchange solid phase extraction (SPE) cartridges were obtained from Phenomenex (Strata, 100 mg/3 mL, USA). Centrifugation was performed using a Refrigerated centrifuge obtained from Eppendorf (5417R, Hong Kong, China).

### Determination of Creatinine

The creatinine concentration inside urine samples were determined by LabAssay Creatinine assay (Wako, Japan). Briefly, urine samples and standards were thawed, deproteinized and centrifuged. The supernatant was separated and reacted with picric acid in alkaline solution to produce tangerine condensate through Jaffe reaction. Quantitation of total creatinine inside samples was made by measurement of absorbance by a Clariostar Monochromator Microplate Reader (BMG Labtech, Hong Kong). Concentrated urine samples which exceeded the calibration points were diluted with water with appropriate dilution factors before sample preparation. Each sample was determined at least twice with relative standard deviation (RSD) of less than 15%.

### Standard Preparation for Determination of Polyamines

Stock solutions (5000 μg/ml) of each polyamine (Put, Spm, Spd) were prepared in water separately. The three stock solutions were mixed and diluted to give an intermediate standard (50 μg/ml), which was then used to prepare a series of working standards with polyamine concentrations of 10, 25, 50, 100, 250, 500, 1000 ng/ml in water. For internal standards, the stock solutions (5000 μg/ml) of each polyamine (Put-d8, Spm-d8, Spd-d8) were prepared in water individually. The three stock solutions were mixed and diluted to give an internal standard (IS) working solution (1 μg/ml) in water. The internal standard (IS), as used in analytical chemistry, refers a chemical substance that is added in a constant amount to samples, the blank and calibration standards in a chemical analysis. This internal standard is then used in data analysis to correct for the loss of analyte during, for example, sample preparation, sample injection and ionization.

### Sample/Standard Pre-Treatment for Determination of Polyamines

The sample preparation procedures followed the method developed in the art with minor modifications. Firstly, urine samples/standards were thawed naturally and centrifuged for 5 minutes at 13000 rpm and room temperature. 120 μl of urine sample/standard supernatant and 60 μl of IS working solution were mixed with 420 μl of water. 550 μl of this well-mixed solution was passed through the SPE cartridges, which had been conditioned and equilibrated with 1 ml of methanol and water, respectively. 450 μl of water was passed through the cartridge afterwards to elute out all polyamines. 400 μl of these SPE-treated samples were then mixed with 100 μl of 10% heptafluorobutyric acid, and the final mixture was ready for instrumental analysis. Concentrated urine samples which exceeded the calibration points were diluted with water with appropriate dilution factors before sample preparation.

### Quality Control Samples for Determination of Polyamines

For each batch of sample analysis, three quality control (QC) working solutions were analysed to verify the accuracy of calibration curves and ensure comparability among batches. The solutions were prepared using analysed control urine samples from our research group. The polyamines concentrations of controls' urine samples were determined and then mixed equally to give a pooled urine sample. Afterwards, three quality control working solutions with different polyamine concentration ranges (low, medium and high) were prepared by mixing this pooled urine sample with standard solutions. For low quality control working solution, the SPE-treated pooled urine sample was mixed with SPE-treated 10 ng/ml standard in a 1:7 ratio. For medium quality control working solution, the SPE-treated, pooled urine samples were mixed with SPE-treated 100 ng/ml standard in a 1:1 ratio. For high quality control working solution, the SPE-treated, pooled urine sample was mixed with SPE-treated 1000 ng/ml standard in a 1:1 ratio.

### Stability Studies

For stability studies, it had previously demonstrated in the art that both the standard mixtures and quality control samples were stable after storing at six hours at room temperature (short-term stability), after storage at −20° C. and −80° C. for two months (long-term stability) and after going three cycles of freezing and thawing before sample preparation (freeze thaw stability). For further verification, both the content of polyamines and creatinine inside both standards and selected urine samples were analysed. It was found that, upon five cycles of freeze and thaw, all the contents were still stable in six months' time when stored at −20° C. For the SPE-treated samples, it was stable for at least two days when stored at 4° C. and up to a year when stored at −20° C.

### Instrumentation and Statistical Analysis

The quantitation of polyamines was performed by Ultra-high Performance Liquid Chromatography coupled with a tandem mass spectrometer made up of two quadrupole mass spectrometers (UPLC-MS/MS). Liquid chromatographic (LC) separation was performed using an Agilent 1290 Infinity Quaternary LC System, while mass spectral analysis was performed using an Agilent 6460 Triple Quadrupole mass spectrometer equipped with an Agilent Jet Stream technology electrospray ionization source. The column used was an Agilent EclipsePlus C18 RRHD (2.1×50 mm, 1.8 μm) protected with an Agilent SB-C18 guard column (2.1×5 mm, 1.8 μm).

The liquid chromatography elution profiles were optimized as follows: Eluent A was water with 0.1% heptafluorobutyric acid, while eluent B was acetonitrile with 0.1% heptafluorobutyric acid. Eluent A was decreased from 95% to 60% in 10 minutes, and from 60% to 10% in 1 minute. Afterwards, the gradient was held constant for 5 minutes. The gradient was then increased from 10% to 95% in 1 minute and held constant for 8 additional minutes. (Total run-time=25 minutes)

The autosampler and column temperatures were set at 4° C. and 35° C., respectively. Injection was achieved by 5-second needle wash in Flush Port mode for 3 times with eluent B. 10 μl was injected each time.

For the source parameter, drying gas (nitrogen) temperature was set as 300° C. with 5 L/min flow rate. Nebulizer pressure was 45 psi. Sheath gas temperature was set as 250° C. with 11 L/min flow rate. Capillary voltage was set as 3500V. For mass detection, scheduled multiple reaction monitoring (MRM) was performed.

The result was calculated using Agilent MassHunter Workstation software. Calibration curves were fitted linearly without any weighing. The correlation coefficients should not be smaller than 0.995. Acceptance values for each calibration points and quality control working solutions were ±30% to ensure accuracy. For precision verification, after every 10-sample injection, a 250 ng/ml standard was injected and checked if it can be reproduced (±15%).

For statistical analysis, the receiving operator characteristic (ROC) curve and the area under the curve (AUC) were obtained by using GraphPad Prism 6 (GraphPad Software, San Diego, Calif., USA). A p-value smaller than 0.05 (two-tailed) was considered to be statistically significant during comparison based on Student's t-test.

Frozen urine and spermine standards were thawed and centrifuged, followed by addition of spermine-(butyl-d8) tetrahydrochloride as internal standard. The diluted samples and working spermine standards (10, 25, 50, 100, 250, 500, 1000 ng/ml) went through solid phase extraction cartridge [Strata® SAX (55 μm, 70 Å), 100 mg] to remove unwanted substance prior to ultra-high performance liquid chromatography coupled with a triple quadrupole mass spectrometer (UPLC-MS/MS) analysis. The determination of creatinine concentration in urine samples was achieved by creatinine enzymatic reagent (BioSystems). Urine spermine values (in mol/g) were normalized with urinary creatinine (in mol/g) to generate normalized spermine (no unit), and all spermine analysis utilized normalized spermine values.

Colleagues involved in urine laboratory work were blinded to clinical and pathology results.

Data interpretation and analyses focused on the core group of patients with a prostate-specific antigen concentration of between 4 to 20 ng/mL and without prior diagnosis of prostate cancer. The risks of prostate cancer and higher-grade prostate cancer (HGPCa) defined as International Society of Urological Pathology (ISUP) grade group (GG) 2 or above cancers, were calculated in different ranges of normalized spermine. Digital rectal examination was categorized to normal and abnormal.

Prostate volume (PV) was estimated by transrectal ultrasound using Ellipsoid formula (height×width×length and divided by 2). Most patients did not have MRI before biopsy and therefore MRI parameters were not included in the analyses.

The prediction of prostate cancer (PCa) and high-grade prostate cancer (HGPCa) by various clinical variables and normalized spermine values was estimated with univariate and multivariate analyses. A Spermine Risk Score was created from a formula derived from multivariate logistic regression analysis. The performance of spermine and different risk models was compared with area under curve (AUC) of receiver operating characteristics (ROC). Decision curve analysis (DCA; see for example, FIG. 12) was performed to compare parameters. Internal validation with bootstrapping was performed. IBM SPSS Statistics for Windows version 25 (IBM Corp., Armonk, N.Y., USA) and R version 3.1.1 (The R foundation for statistical computing, Vienna, Austria) was used for statistical analyses. A two-sided p value of <0.05 was considered significant.

1. A method of determining the risk of developing prostate cancer in a subject or determining whether a subject suffers from prostate cancer, the method comprising
a. measuring the level of one or more polyamines in a fluid sample obtained from the subject,
b. measuring at a variable selected from the group consisting of age, prostate volume (PV), prostate-specific antigen (PSA), digital rectal examination (DRE), and combinations thereof;
c. comparing the level of the one of more polyamines and the at least one variable to a control;
wherein a decrease or increase in the level of the one or more polyamines compared to the control indicates that the subject is at risk of developing, or suffers from, prostate cancer;
wherein a decrease in prostate volume and/or an increase in prostate-specific antigen (PSA) compared to the control indicates that the subject is at risk of developing, or suffers from, prostate cancer;
wherein a positive digital rectal examination result indicates that the subject is at risk of developing, or suffers from, prostate cancer;
wherein the one or more polyamines is selected from the group consisting of spermine, spermidine and putrescine.

2. The method of claim 1, wherein the polyamine is spermine and putrescine.

3. The method of claim 1, wherein the polyamine is spermine.

4. The method of claim 1, wherein the level of polyamines is normalised.

5. The method of claim 4, wherein the normalisation is done with creatinine.

6. The method of claim 1, wherein the control is a subject with a prostate cancer of ISUP<2, or a cancer-free subject.

7. The method of claim 1, wherein the subject had been determined to have a prostate-specific antigen (PSA) concentration of at least 4 ng/ml prior to preforming the method.

8. The method of claim 7, wherein the prostate-specific antigen (PSA) concentration of between 4 ng/ml and 20 ng/ml.

9. The method of claim 1, wherein the fluid sample is selected from the group consisting of cellular and non-cellular components of amniotic fluid, breast milk, bronchial lavage, cerebrospinal fluid, colostrum, interstitial fluid, peritoneal fluids, pleural fluid, saliva, seminal fluid, urine, tears, whole blood, plasma, serum plasma, and serum.

10. The method of claim 9, wherein the fluid sample is selected from the group consisting of urine, whole blood, plasma, serum plasma, and serum.

11. The method of claim 1, wherein if the subject is identified as not being at risk of developing or suffering from prostate cancer, the subject is not subjected to further testing.

12. The method of claim 1, wherein if the subject is identified as being at risk of developing or suffering from prostate cancer, the method further comprises an action selected from the group consisting of monitoring, surgery, surgical castration, medical castration, and combinations thereof.

13. A method of determining the risk of developing prostate cancer in a subject or determining whether a subject suffers from prostate cancer, the method comprising
d. measuring the level of one or more polyamines in a fluid sample obtained from the subject,
e. measuring a variable selected from the group consisting of age, prostate volume (PV), prostate-specific antigen (PSA), digital rectal examination (DRE), and combinations thereof;
f. obtaining a score value based on the level of the one or more polyamines measured in step d. and the at least one variable measured in step e. to predict the likelihood of the subject developing or having prostate cancer;
wherein an increase in score value indicates that the subject is at an increased risk of developing, or suffers from, prostate cancer.

14. The method of claim 13, wherein the score value is calculated using i) area under the curve (AUC) of receiver operating characteristics (ROC), logistic regression, Akaike information criterion (AIC), and Bayesian information criterion (BIC); ii) p-value based on a test selected from the group consisting of student's t-test, Mann-Whitney U test, Chi-square test, two-sided t-test; and/or iii) a classification algorithm selected from the group consisting of support vector machine algorithm, logistic regression algorithm, multinomial logistic regression algorithm, Fisher's linear discriminant algorithm, quadratic classifier algorithm, perceptron algorithm, k-nearest neighbour's algorithm, artificial neural network algorithm, random forests algorithm, decision tree algorithm, naive Bayes algorithm, adaptive Bayes network algorithm, and ensemble learning method combining multiple learning algorithms.

15. The method of claim 14, wherein the classification algorithm is pre-trained using the level of the one or more polyamines of the control, and at least one variable selected from the group consisting of age, prostate volume (PV), prostate-specific antigen (PSA), digital rectal examination (DRE), and combinations thereof, of the control.

16. The method of 14, wherein the classification algorithm compares the level of the one or more polyamines present in the sample obtained from the subject and at least one variable, with that of the control, and returns a mathematical score that identifies the likelihood of the subject to belonging to the control or not.

17. A kit for use according to the method of claim 1.
